(19) United States
(12) Patent Application Publication
(10) Pub. No.: US 2005/0112161 A1
(43) Pub. Date: May 26, 2005

(54) Transparent oil gelling system

(76) Inventors: Dexin Luo, Fresh Meadows, NY (US); Tian Xiang Wang, Dix Hills, NY (US); Tatyana Tabakman, Brooklyn, NY (US); Shahan Nazar, Garden City, NY (US); Steve Hasher, Blue Point, NY (US); Joseph Gubernick, New York, NY (US)

Correspondence Address: THE ESTEE LAUDER COS, INC, 125 PINELAWN ROAD, MELVILLE, NY, 11747, US

(21) Appl. No.: 10/985,781

(22) Filed: Nov. 10, 2004

Related U.S. Application Data
(60) Provisional application No. 60/519,583, filed on Nov. 13, 2003.

(51) Int. Cl. A61K 7/32; A61K 7/06; A61K 7/11

(52) U.S. Cl. 424/401; 424/70.13

(57) ABSTRACT

The invention relates to a gellant system comprising gellant effective amounts of at least one silica and at least one sugar fatty acid ester. The gellant system of the invention is useful in gelling polar oils to produce transparent or translucent gels useful in topical compositions.

### FIELD OF THE INVENTION

The invention relates to the field of cosmetics. More specifically, the invention relates to a gelling system for cosmetic oils.

### BACKGROUND OF THE INVENTION

When women are asked what cosmetic item they simply could not be without, a typically high percentage reply that lip color is an essential item of their beauty wardrobe. With a product that is so widely used by the cosmetic-purchasing public, it is not surprising that there is a continuing demand for new and innovative product types. Among the current trends for lip products are those that remain in place for several hours without the need for reapplying, as well as those that provide a high level of gloss and shine. In many cases, it is highly desirable to combine these two characteristics. While there is a considerable variety of products of these types currently available, the commercially available products frequently suffer from certain drawbacks with a similar root problem. For example, the long-wearing products on their own can be drying and uncomfortable on the lips, with a dull finish, thereby requiring a moisturizing topcoat to be applied over them. Such topcoats, however, are difficult to formulate, since they need to be sufficiently incompatible with the base coat, which is frequently based on non-polar hydrocarbons and silicones, to prevent interference with the base coat's wear, and at the same time, are preferably transparent to give the best level of glossy appearance to the lips. Typically, however, the currently available topcoat products are opaque sticks that produce a small amount of shine. Similarly, with lip gloss products intended to provide high shine, it is difficult to achieve the desired level of transparent gloss without producing a product that is too liquid or oily. The problem in producing the perfect product lies in the difficulty of gelling the cosmetic oils that constitute the backbone of the majority of lip products. The most commonly used oils are typically polar, and achieving the desired level of viscosity and clarity is a complex matter. Although it is of course possible to gel such oils, the usual viscosifying agents employed are waxes and/or clays; the end product achieved with the use of such materials is typically either too opaque or else less viscous and more oily-feeling that would be desired. Thus there continues to be a need for a soft liquid gel-type product that does not convey an oily feeling on the lips, yet provides a significant level of transparency that will improve the gloss and shine of the final product. The present invention now provides a solution to this need.

### SUMMARY OF THE INVENTION

The present invention relates to a gel-type topical composition comprising at least one cosmetically acceptable polar oil, and a gellant for the oil comprising at least one silica and at least one sugar fatty acid ester. The invention also relates to a method of gelling a polar cosmetically acceptable oil which comprises adding to the oil at least one silica and at least one sugar fatty acid ester, in an amount and under conditions sufficient to gel the oil. The compositions of the invention are soft, lubricious, transparent gels, viscous liquids or pastes that are particularly well adapted for use as a glossy top coat for a transfer resistant base coat or as a high shine lip gloss on its own. The invention also comprises a method of conferring shine to a skin surface which comprises applying to the skin surface a composition comprising at least one polar oil and a gellant comprising a complex of at least one silica and at least one sugar fatty acid ester.

### DETAILED DESCRIPTION OF THE INVENTION

The base of the composition is a cosmetically acceptable polar oil. The term “cosmetically acceptable” oil means one that is industry-accepted as safe for use on the skin surface to which the product is intended to be applied. Polar oils are frequent components of cosmetic compositions, and are distinguished from non-polar oils, such as hydrocarbons, by their relative lack of hydrophobicity. Polar oils typically contain heteroatoms, with higher electron negativity than carbon, e.g., alcohol residues, or an ester or triglyceride component. Examples of useful polar oils include, but are not limited to, vegetable oils and triglycerides (including hydrogenated liquid vegetable oils), such as castor oil, coconut oil, corn oil, jojoba oil, cottonseed oil, soybean oil, walnut oil, wheat germ oil, peach kernel oil, olive oil, peanut oil, sunflower seed oil, palm kernel oil, calendula oil, illipe butter, shea butter and caprylic/capric triglycerides; esters having the formula RCO—OR′ wherein RCO represents a carboxylic acid radical and OR′ represents an alcohol residue, such as isodecyl neopentanoate, tridecyl octanoate, cetyl palmitate, cetyl octanoate, cetyl stearate, cetyl myristate, isopropyl palmitate, isopropyl myristate, polyglyceryl-2-isostearate, neopentyl glycol distearate, isodecyl oleate, decyl isostearate, diisopropyl sebacate, PEG-4 diheptanoate, dioctyl malate, and isohexyl neopentanoate; polyol fatty acid polyesters, e.g., fatty acid polyesters derived from aliphatic or aromatic polyols that have at least 4 free hydroxyl groups, of which at least 80% of these free hydroxy groups are then esterified with one or more fatty acids having from 8 to 22 carbon atoms, preferably polyol fatty acid polyesters that are derived from sugar polyols that comprise mono-, di, and polysaccharides or sugar alcohols (see U.S. Pat. No. 6,555,097, the contents of which are incorporated herein by reference); and fatty alcohols, such as lanolin alcohol, cetyl alcohol, isocetyl alcohol or oleyl alcohol. There may be a single polar oil, or a combination of polar oils, employed in the composition. The polar oils will normally constitute from about 10 to about 99% by weight of the composition, more preferably from about 30 to about 95% of the composition. When using polar oils that are solid or semi-solid, lower quantities, e.g., about 10% or less, are preferred to retain the transparent or translucent effect.

Polar oils, particularly non-silicone polar oils, are typically difficult to gel and retain the desired clarity. However, it has been discovered that, in accordance with the present invention, key elements in the successfully gelling polar oils, while retaining considerable clarity, are a combination of at least one silica and at least one sugar fatty acid ester or ether. Although neither of these materials used alone will provide the desired gelling effect, when used together, as described in more detail below, the result is a high viscosity, transparent or translucent liquid gel with excellent aesthetics for application to the lips or other skin surfaces where a clear shine or gloss is desired. While not wishing to be bound by any particular theory, it is believed that the hydroxide groups of the sucrose fatty esters interact via hydrogen bonding with the hydroxide groups on the surface of the silica particles, while the fatty acid portion of the ester molecule and the remaining hydroxide groups can interact with the polar oil components of the composition. Thus, perhaps, the suspended silica serves as a physical cross-linking center while the fatty ester acts as a network spacer to form a gel phase. The success in this combination is particularly surprising in view of the general lack of success previously in producing a clear gelled polar oil.

Any silica particle can be used in the gelling process, provided the particle is not fully surface-coated. Use of partially coated products, while possible, will result in the need to use higher levels of silica to achieve the desired effect. The amount of silica used is not particularly critical, and can be employed in an amount of up to about 40% by weight of the composition, although the higher levels will produce a drier, less aesthetically pleasing product, and the higher levels necessary with particles having less exposed surface area do not viscosiiy as well as lower levels. The preferred silica employed in the gellant component is a fumed silica. By “fumed silica” it is meant those high-surface area powdered silicas prepared by a pyrogenic process, e.g., during burning silicon tetrachloride in air (i.e., by the flame hydrolysis of silicon tetrachloride) and has a purity of 99.8% or greater. In this process, submicron sized molten spheres of silica collide and fuse to form three dimensional, branched, chain-like aggregates, of approximately 0.1 to 0.5 microns in length. Cooling takes place very quickly, limiting the particle growth and ensuring the fumed silica is amorphous. Fumed silicas are available in untreated form, or with a surface treatment to render the silica more polar or non-polar. Although any type can be used, preferably the fumed silica used in the present invention is untreated, or at most partially treated. A fumed silica fully coated with non-polar materials will not provide the desired effect, although a silica coated with polar material, such as dimethicone copolyol, may provide some utility. The surface area of the fumed silica is preferably between about 90 to about 380m²/g, and most preferably is between about 200 to about 380m²/g. A particularly useful fumed silica is commercially available from Cabot Corporation under the trade name Cab-O-Sil M-5. As a guideline, for an aesthetically pleasing product, a fumed silica is employed in an amount of about 0.2 to about 10% by weight, preferably about 1 to about 5%, of the total composition.

The sugar fatty acid ester employed in the invention is a compound obtained by reacting a saturated or unsaturated C12-C22 fatty acid, preferably C16-C20, with a sugar or alkylsugar in which the alkyl group contains from 1 to 8 carbon atoms. The sugar is preferably a mono- or oligosaccharide. Examples of useful mono- or oligosaccharides include, but are not limited to, glucose, sucrose, galactose, fructose, lactose, mannose, maltose, trehalose, melibiose, raffinose, or ribose. A preferred sugar fatty ester is a fatty ester of glucose or allylglucose. The fatty acid esters of alkylglucose are ethers of glucose in which the alkyl chain comprises from 1 to 8 carbon, atoms, preferably 1-4 carbon atoms. The preferred ester may contain a mixture of mono-, di-, tri- and tetraester derivatives with a proportion which may be of at least 50% by weight of mono- and diester derivatives and usually not exceeding 95% by weight of monoester derivatives relative to the total weight of the mixture. Examples of sugar fatty esters that may be used in the invention include, but are not limited to, sucrose monolaurate, glucose palmitate, alkylglucose sesquistearates, for instance methylglucose sesquistearate and alkylglucose palmitates, for instance methylglucose palmitate or ethylglucose palmitate, as well as the PEG or PPG derivatives of such compounds, for example, PEG-20 methyl glucose sesquistearate. Such compounds are widely available commercially, e.g., under the tradenames Glucate™, Glucam™, and Glucamate™ (Amerchol), Grillocose™ (Grillo-Werke), and Antil™(Goldschmidt). The amount of ester used in the composition will be from about 0.1 to about 10%, preferably about 0.5 to about 5%, by weight of the composition.

The viscosity of the end product is dependent upon the ratio of the amount of silica to the amount of sugar fatty acid ester. Overall the ratio of these materials will range from about 10:0.5 to 0.5:5.0 silica to ester, with the lower amounts of the silica producing a lower viscosity product, and higher amounts of the silica producing a higher viscosity product. A product having a ratio of about 6:1 to 1:5, more preferably about 4.0:1.0-1:1, most preferably about 3:1-1:1, silica: ester is particularly preferred. The viscosity is also ultimately affected by the amount of gellant used relative to the amount of polar oil, with a higher viscosity achieved by a higher amount of gelling components. The viscosity is also affected by the polarity of the oils used, as the silica is more readily suspended in a more polar oil, presumably due to the interaction of the hydroxyl groups of the silica and the polar groups on the oils, so that a well-suspended silica can be used at smaller amounts than a silica that is not so readily suspended. The clarity of the gel can also be influenced by the amount of sugar ester used; although a more opaque gel still provides a gel system with unique and aesthetically pleasing properties, a transparent or translucent character is still preferred. Therefore, to maintain the clarity of the final product, it preferred that no more than about 3% ester be employed as a whole in the formulation. Clarity is also enhanced by ensuring that the silica is well-dispersed in the ester, which can be achieved by extending homogenization time.

The use of the combination of silica and sugar fatty acid ester in gelling the oil is relatively simple. The silica and oil components are mixed together until smooth. Any additional components of the composition, except for the ester, are then added and blended, while the temperature is raised to about 10° C. above the melting point of the sugar ester. The ester is then added to the mixture, mixed until dissolved, with continued mixing under the same temperature and mixing conditions, for several minutes (e.g., 30 minutes) until network formation is complete, which typically occurs at the point when all the components are dissolved. The product is then lowered to room temperature or to the desired pouring temperature.

Although the components mentioned above represent the minimum required to produce an acceptable gel, it may be desirable to incorporate additional components into the composition to provide added functionality. A particularly preferred component is one or more hydrocarbon polymers, particularly high viscosity liquid or paste polymers having a molecular weight of between about 400 to 6000 Daltons. While higher molecular weights can be used, they will tend to reduce the clarity of the product, resulting in a more translucent rather than transparent appearance. The hydrocarbon polymers are useful in conferring some additional viscosity, but also contribute to the feel of the product. In general, they confer a greater amount of tack to the composition, which in turn results in longer wear. Examples of materials useful for this purpose are linear or branched polybutene, polyisobutene, polyethylene, polydecene, hydrogenated derivatives thereof, and copolymers thereof, and mixtures of the foregoing. Particularly preferred is a hydrogenated polyisobutene. If used, the hydrocarbon polymer is used in an amount of about 1 to about 70%, preferably about 5 to about 50%, by weight of the composition.

An additional optional component may be an oil soluble film-forming agent, for example, acrylate polymers, polyurethanes, linear or branched polyhydrocarbon film-formers. The film-former is typically used in an amount of from 0 to about 20% by weight.

The composition may also contain oil soluble active agents and skin conditioning agents. Non-limiting examples of these materials include antioxidants, ceramides, fatty acids, sunscreens, emollients, oil soluble vitamins and plant extracts, and the like. When used as the oil phase of an emulsion, the composition can also contain water soluble actives.

Depending upon its intended final use, the product may also contain a colorant. Any type of pigment, provided it is acceptable for use in the area to which the product will be applied, and with or without surface treatment, can be used in the product of the invention: examples of useful pigments include iron oxides (yellow, red, brown or black), titanium dioxide (white), zinc oxide, chrome oxide (green), chrome hydrate (green), ultramarines, manganese violet, ferric ferrocyanide, carmine 40, ferric ammonium ferrocyanide, or combinations thereof. Interference pigments, which are thin platelike layered particles having a high refractive index, which, at a certain thickness, produce interference colors, resulting from the interference of typically two, but occasionally more, light reflections, from different layers of the plate, can also be added to provide a pearlescence to the product, is such is desired. The composition may also contain one or more types of cosmetically acceptable glitter, i.e., particles of transparent or colored, solid organic materials, such as poly(ethylene terephthalate), polymethacrylate, and poly(vinylbutyral), particles of metal, or particles of metal coated film or paper.

Organic pigments may also optionally be included; these include natural colorants and synthetic monomeric and polymeric colorants. Exemplary are phthalocyanine blue and green pigment, diarylide yellow and orange pigments, and azo-type red and yellow pigments such as toluidine red, litho red, naphthol red and brown pigments. Also useful are lakes, which are pigments formed by the precipitation and absorption of organic dyes on an insoluble base, such as alumina, barium, or calcium hydrates. Particularly preferred lakes are primary FD&C or D&C Lakes and blends thereof. Stains, such as bromo dyes and fluorescein dyes can also be employed. Pigments when used are typically present in an amount of about 0.1 to about 30%, preferably about 0.1 to about 20%, by weight of the composition.

The gel system of the present invention can be used in the same manner as other gelling systems are in cosmetic compositions. Typical uses for gellant systems in cosmetics are structural support, prevention of liquid from flowing, controlled release of included agents, and the like. The gel system of the present invention provides all these functionalities, and may be used as a thickening component of any polar oil containing cosmetic, such as skin care creams, lotions, sticks or serums, as well as in color cosmetics, such as eyeshadows, blushes, mascaras, lipsticks, and the like. This polar-oil based gel can be used on its own, in an anhydrous product, or it can be used to thicken the oil phase of water and oil emulsion systems. The gel system is very useful as the base of a lip product. It has particular advantage when used in a moisturizing or shine-conferring top coat to a hydrocarbon- or silicone-based base coat (including, but not limited to, that described in U.S. Pat. No. 6,340,466 or 6,019,962, contents incorporated herein by reference) that may be matte, long-wearing, transfer resistant or drying, and may improve the wear of long-wearing or transfer resistant products. It may on its own also serve as a lipgloss, lip balm and soft gel lip color. In this context, it is useful in avoiding feathering that is so common in high shine lip products, as well as enhancing the wear and transfer resistance of the product itself as well as any transfer resistant base coat with which it is used.

The invention is further illustrated by the following non-limiting examples.

### EXAMPLE1

This example illustrates formulations of the compositions of the invention.

A.

| Material | Weight percent |
|---|---|
| Castor oil | 33.21 |
| Fumed silica | 1.79 |
| Sucrose acetate dibutyrate | 62.00 |
| Isopropylparaben/isobutylparaben/ | 0.10 |
| butylparaben | |
| Polyglyceryl-2- | 2.00 |
| diisosteaerate/IPDI copolymer | |
| Vitamin E | 0.10 |
| Methyl glucose sesquistearate | 0.80 |

B.

| Material | Weight percent |
|---|---|
| Sucrose polycottonseedate | 69.60 |
| Fumed silica | 2.25 |
| BHT | 0.05 |
| Isopropylparaben/isobutylparaben/ | 0.10 |
| butylparaben | |
| Hydrogenated polyisobutene | 27.00 |
| Methyl glucose sesquistearate | 1.00 |

1. A gellant system comprising gellant effective amounts of at least one silica and at least one sugar fatty acid ester.

2. The system of claim 1 in which the silica is a fumed silica.

3. The system of claim 1 in which the ester is a reaction product of a saturated or unsaturated C12-C22 fatty acid with a sugar or a C1-C8 alkyl sugar.

4. The system of claim 3 in which the fatty acid is a C16-C20 fatty acid.

5. The system of claim 3 in which the sugar is a mono or oligosaccharide.

6. The system of claim 5 in which the ester is selected from the group consisting of sucrose monolaurate, glucose palmitate, alkylglucose sesquistearates, alkylglucose palmitates, PEG derivatives thereof, PPG derivatives thereof, and mixtures of any two or more of these esters.

7. The system of claim 1 in which the ratio of silica to ester is about 10:0.05 to about 0.5:5.

8. The system of claim 1 in which the ratio of silica to ester is about 6:1 to about 1:5.

9. The system of claim 1 in which the ratio of silica to ester is about 3:1 to about 1:1.

10. A composition for topical application to the skin comprising at least one polar oil and a gellant system comprising gellant effective amounts of at least one silica and at least one sugar fatty acid ester.

11. The composition of claim 10 in which the polar oil is selected from the group consisting of vegetable oils; triglycerides; esters having the formula RCO—OR′, in which RCO is a carboxylic acid radical and OR′ is an alcohol residue; polyol fatty acid polyesters; fatty alcohols, and mixtures thereof.

12. The composition of claim 10 in which the silica is a fumed silica.

13. The composition of claim 10 in which the ester is selected from the group consisting of sucrose monolaurate, glucose palmitate, alkylglucose sesquistearates, alkylglucose palmitates, PEG derivatives thereof, PPG derivatives thereof, and mixtures of any two or more of these esters.

14. A composition for topical application to the skin comprising at least one polar oil selected from the group consisting of vegetable oils; triglycerides; esters having the formula RCO—OR′, in which RCO is a carboxylic acid radical and OR′ is an alcohol residue; polyol fatty acid polyesters; fatty alcohols, and mixtures thereof; and a gellant system comprising gellant effective amounts of at least one fumed silica and at least one sugar fatty acid ester selected from the group consisting of sucrose monolaurate, glucose palmitate, alkylglucose sesquistearates, alkylglucose palmitates, PEG derivatives thereof, PPG derivatives thereof, and mixtures of any two or more of these esters.

15. The composition of claim 10 in which the ratio of silica to ester is about 6:1 to 1:5.

16. The composition of claim 10 which also comprises a hydrocarbon polymer having a molecular weight from about 400 to about 6000.

17. The composition of claim 14 which is a composition for application to the lips.

18. A two part system for application of color and/or shine to the lips comprising (a) a hydrocarbon or silicone-based base coat, and (b) a top coat comprising a polar oil and a gellant system comprising gellant effective amounts of silica and a sugar fatty acid ester.

19. The system of claim 16 in which the top coat comprises at least one polar oil selected from the group consisting of vegetable oils; triglycerides; esters having the formula RCO—OR′, in which RCO is a carboxylic acid radical and OR′ is an alcohol residue; polyol fatty acid polyesters; fatty alcohols, and mixtures thereof; and a gellant system comprising gellant effective amounts of at least one fumed silica and at least one sugar fatty acid ester selected from the group consisting of sucrose monolaurate, glucose palmitate, alkylglucose sesquistearates, alkylglucose palmitates, PEG derivatives thereof, PPG derivatives thereof, and mixtures of any two or more of these esters.

20. The system of claim 18 in which the base coat is long-wearing or transfer resistant.
